(19) United States
(12) Patent Application Publication
(10) Pub. No.: US 2023/0324387 A1
(43) Pub. Date: Oct. 12, 2023

(54) METHOD FOR DETECTION OF ANTIGEN-SPECIFIC ANTIBODY

(71) Applicant: The Johns Hopkins University, Baltimore, MD (US)

(72) Inventors: Stephen J. Gould, Baltimore, MD (US); Chenxu Guo, Baltimore, MD (US); Shang-Jui Tsai, Baltimore, MD (US)

(21) Appl. No.: 18/033,925

(22) PCT Filed: Nov. 1, 2021
(86) PCT No.: PCT/US2021/057611

§ 371 Date: Apr. 26, 2023

Related U.S. Application Data
(60) Provisional application No. 63/108,833, filed on Nov. 2, 2020.

(51) Int. Cl. G01N 33/569; G01N 33/68

(52) U.S. Cl. CPC G01N 33/56983; G01N 33/6854; G01N 2333/165; G01N 2469/20

(57) ABSTRACT

The present disclosure provides methods for the detection of target specific antibodies in samples. The methods include the detection of pathogen-specific antibodies, such as SARS-CoV-2 specific antibodies. Also included is a kit for use for the detection of pathogen specific antibodies in a sample. In one aspect, the disclosure provides a method of detecting the presence of a target specific antibody in a sample by (i) contacting the sample with a test cell comprising one or more exogenous nucleic acid sequences encoding one or more target proteins; and (ii) detecting the presence of the target specific antibody in the sample by contacting the immune complex of (i) with an anti-immunoglobulin (Ig) antibody, and detecting the anti-immunoglobulin (Ig) antibody, thereby detecting the presence of a target specific antibody.

### CROSS REFERENCE TO RELATED APPLICATIONS

This application claims the benefit of priority under 35 U.S.C. §119(e) of U.S. Provisional Application No. 63/108,833, filed Nov. 2, 2020 which is herein incorporated by reference in its entirety.

### INCORPORATION OF SEQUENCE LISTING

The material in the accompanying sequence listing is hereby incorporated by reference into this application. The accompanying sequence listing text file, name JHU4180_1WO_SL.txt was created on Oct. 27, 2021, and is 1 kb. The file can be assessed using Microsoft Word on a computer that uses Window OS.

### BACKGROUND OF THE INVENTION

### Field of the Invention

The present invention relates generally to detection of antigens and more specifically to a serology test for antigens, including pathogens such as severe acute respiratory syndrome coronavirus 2 (SARS-CoV-2).

### Background Information

Severe acute respiratory syndrome coronavirus 2 (SARS-CoV-2) is the virus strain responsible for coronavirus disease 2019 (COVID-19), a respiratory illness which has been designated as a pandemic disease by the World Health Organization (WHO).

SARS-CoV-2 belongs to the broad family of coronaviruses. It is a positive-sense single-stranded RNA (+ssRNA) virus, with a single linear RNA segment. Other coronaviruses are capable of causing illnesses ranging from the common cold to more severe diseases such as Middle East respiratory syndrome (MERS, fatality rate ~34%). It is the seventh known coronavirus to infect people, after 229E, NL63, OC43, HKU1, MERS-CoV, and the original SARS-CoV. Taxonomically, SARS-CoV-2 is a strain of severe acute respiratory syndrome-related coronavirus (SARSr-CoV). As the SARS-related coronavirus strain implicated in the 2003 SARS outbreak, SARS-CoV-2 is a member of the subgenus Sarbecovirus (beta-CoV lineage B). Its RNA sequence is approximately 30,000 bases in length, and uniquely incorporates a polybasic cleavage site, known to increase pathogenicity and transmissibility in other viruses.

As of Oct. 22, 2020, there have been 41,584,690 total confirmed cases of SARS-CoV-2 infection in the ongoing pandemic, with a total number of 1,135,767 deaths attributed to the virus. While the majority of cases result in mild symptoms, including fever, cough, fatigue, shortness of breath, and both loss of smell and taste, some progress to viral pneumonia, multi-organ failure, or cytokine storm. Older adults and people who have severe underlying medical conditions like heart or lung disease or diabetes seem to be at higher risk for developing more serious complications from COVID-19 illness.

The virus primarily spreads between people through close contact and via respiratory droplets produced from coughs or sneezes. People may also become infected by touching a contaminated surface and then touching their face. It mainly enters human cells by binding to the receptor angiotensin converting enzyme 2 (ACE2). Epidemiologic studies estimate that each infection results in 1.4 to 3.9 new ones when no members of the community are immune and no preventive measures taken.

SARS-CoV-2 is an enveloped virus responsible for the COVID-19 pandemic. This beta-coronavirus encodes more than 25 distinct proteins that act to reprogram host cells for virus production. SARS-CoV-2 virions that emanate from infected cells are extracellular vesicles that are comprised of a protein-rich lipid bilayer enriched in four structural proteins of the virus, nucleocapsid (N), spike (S), membrane (M), and envelope (E). S, M and E are integral proteins of the virus membrane and serve to drive virion budding while also recruiting the N protein and the viral genomic RNA into nascent virions. Studies of other coronaviruses have established that co-expression of these four structural proteins is sufficient to drive the formation of virus-like particles (VLPs), and this also appears to be the case for SARS-CoV-2.

The S protein is the largest of the SARS-CoV-2 structural proteins. The 1274 amino acid-long S protein contains an N-terminal signal sequence, a large extracellular domain with receptor-binding and membrane-fusion activities, and a hydrophobic transmembrane domain just upstream of its short, cytoplasmically-oriented, carboxy-terminal tail. The synthesis of the S protein occurs on the surface of the endoplasmic reticulum (ER) where the protein is co-translationally translocated into the lumen and membrane of the ER, with only its short, carboxy-terminal tail remaining on the cytoplasmic side of the membrane. The extracellular domain of spike is subject to extensive post-translational modification, including removal of its N-terminal signal sequence, formation of intramolecular disulfide bonds, extensive glycosylation, and the adoption of multiple conformational states. Spike may traffic from the ER to the Golgi, and the co-expression of the M and E proteins may enhance spike accumulation at sites of coronavirus assembly, which has been postulated to occur in the ER-Golgi intermediate compartment (ERGIC), Golgi, and/or lysosome.

At some point in its biogenesis, spike encounters one or more proteases that cleave it into an N-terminal S1 fragment and a C-terminal S2 fragments, with the resulting two polypeptides held together only by non-covalent interactions. This cleavage event is essential for virus-cell fusion, but is not always completed during virion biogenesis, resulting in the production of viruses with variable amounts of cleaved and uncleaved spike proteins, as well as variable loss of the S1 protein from the virus particle. Once released, the spike protein mediates the key events of virus-cell interaction, with the S1 region of the protein binding angiotensin converting enzyme-2 (ACE2), the primary SARS-CoV-2 receptor, and the S2 region subsequently mediating fusion of the viral and cellular membranes in a process that requires prior proteolysis at the S1/S2 cleavage site, whether by furin or other proteases in the virus producing cell, TMRSS2 at the surface of the target cell, or lysosomal proteases following viral endocytosis.

A variant of SARS-CoV-2 has arisen that displays many hallmarks of increased viral fitness, including enhanced rate of transmission in diverse human populations, elevated viral load in vivo, and enhanced viral titer in vitro. This variant strain of SARS-CoV-2 contains a mutation in the spike gene, D614G, that changes the aspartate at position 614 to a glycine residue. The S^{D614G} protein appears to be more abundant on virus particles and to lose less free S1 fragment, relative to the S protein encoded by the original Wuhan-1 isolate of SARS-CoV-2 (S^{W1}).

The current standard method of diagnosis is by real-time reverse transcription polymerase chain reaction (rRT-PCR) from a nasopharyngeal swab. Chest CT imaging may also be helpful for diagnosis in individuals where there is a high suspicion of infection based on symptoms and risk factors; however, guidelines do not recommend using it for routine screening. In the absence of specific treatment or vaccine for COVID-19, infected subjects are only managed with supportive care, which may include fluid therapy, oxygen support, and supporting other affected vital organs.

There is therefore an unmet need for tools to detect SARS-CoV-2 infection and efficient immunization to prevent SARS-CoV-2 infection.

### SUMMARY OF THE INVENTION

The present invention is based on the seminal discovery that the presence of target specific antibodies can be detected in a sample using a test cell including exogenous nucleic acid sequences encoding one or more target proteins.

In one embodiment, the invention provides a method of detecting the presence of a target specific antibody in a sample including: (i) contacting the sample with a test cell including one or more exogenous nucleic acid sequences encoding one or more target proteins; and (ii) detecting the presence of the target specific antibody in the sample by contacting an immune complex of (i) with an anti-immunoglobulin (Ig) antibody, and detecting the anti-immunoglobulin (Ig) antibody, thereby detecting the presence of a target specific antibody.

In one aspect, the sample is further contacted with a control cell, wherein a target specific antibody present in the sample forms an immune complex with the one or more target proteins expressed by the test cell. In another aspect, detecting the presence of the target specific antibody includes contacting the immune complex with an anti-immunoglobulin (Ig) antibody. In one aspect, the control cell does not express a target protein. In one aspect, the target protein is a pathogen protein. In another aspect, the pathogen is a virus or a bacteria. In some aspects, the pathogen is SARS-CoV-2 virus. In one aspect, the pathogen protein is selected from SARS-CoV-2 spike (S), nucleocapsid (N), membrane (M), and envelope (E) proteins. In another aspect, the SARS-CoV-2 S protein is an S^{W1} protein, an S^{D614G} protein or an S** protein. In one aspect, the test cell and the control cell are an adherent fixed and permeabilized cell, a suspension of fixed and permeabilized cells, or a cell lysate coated on a surface of the support. In another aspect, the sample is selected from the group consisting of blood, plasma, serum, urine, saliva, sweat, cerebrospinal fluid (CSF), an antibody and a labeled antibody. In one aspect, the anti-Ig antibody is an anti IgG, IgM, or IgA antibody, or a combination thereof. In another aspect, the anti-Ig antibody is detectably labeled. In one aspect, the anti-IgG, -IgM, or -IgA antibody detects an immune complex including a target protein expressed by the test cell and an anti-target specific antibody present in the sample. In some aspects the immune complex is detected by immunofluorescent microscopy, flow cytometry, or enzyme-linked immunosorbent assay (ELISA). In one aspect, detecting an anti-target specific antibody in the sample includes detecting the detectably labeled anti-Ig antibody and the detectable protein in the test cell but not in the control cell. In another aspect, detecting an anti-pathogen specific antibody in the sample indicates that the subject is infected by the pathogen, has developed an immunity against a pathogen related disease and/or infection, and/or is vaccinated against the pathogen related disease and/or infection. In one aspect, detecting the detectably labeled anti-Ig antibody alone in a test cell, detecting the detectable protein alone in the test cell, or detecting the detectably labeled anti-Ig antibody and/or the detectable protein in a control cell indicates an absence of anti-pathogen specific antibody in the sample. In another aspect, the anti-pathogen specific antibody is an anti-SARS-CoV-2 antibody selected from an anti- S protein antibody, an anti- N protein antibody, an anti- M protein antibody, an anti- E protein antibody or a combination thereof. In some aspects, the anti-pathogen specific antibody detected is an anti-S protein antibody. In other aspects, an anti-N antibody is further detected. In some aspects, an anti-M antibody is further detected. In one aspect, the test cell and control cell are present in a ratio of about 9:1 to 4:1. In another aspect, the one or more exogenous nucleic acid sequences further encode an inducible promoter. In one aspect, a sub-cellular localization of the target specific antibody is further detected. In one aspect, detecting the subcellular localization of the target specific antibody includes contacting the cells with one or more organelle-specific antibodies and determining co-localization of the target specific antibody and the organelle-specific antibodies. In some aspects, the one or more organelle-specific antibodies is an anti-lysosome marker antibody, an anti-Golgi marker antibody, an anti-ER-Golgi-intermediate compartment antibody, a plasma membrane antibody, or a combination thereof. In some aspects the anti-lysosome marker antibody binds to a marker selected from the group consisting of Lamp1, Lamp2, CD63/Lamp3 and mTOR. In other aspects, the sample is a plasma sample and the ratio of the plasma sample to the anti-lysosome antibody is about 1:50 to 1:100,000. In some aspects, the Golgi marker is GM130. In one aspect, detecting the presence of the target specific antibody includes detecting the target specific antibody directly or indirectly by immunofluorescence microscopy. In other aspects, a subcellular localization of a target specific antibody is further detected in a second test cell, wherein the target protein in a first test cell and in the second test cell localize differently. In one aspect, the first and second target proteins are mutant variants of one another. In other aspects, the sample is collected from a subject, the target protein is SARS-CoV-2 Spike protein a S^{W1} protein, a S^{D614G} protein or a S** protein, and detecting sub-cellular localization of the target specific antibody includes the detection of localization to the Golgi, wherein localization to the Golgi indicates the presence of SARS-CoV-2 neutralizing antibodies in the sample. In another aspect, the target specific antibody is detectably labeled.

In another embodiment, the invention provides an isolated peptide of

SEQ ID NO: 1 DSEPVLKGVKLHYT.

In an additional embodiment, the invention provides an antibody that specifically binds to the peptide of SEQ ID NO: 1.

In one embodiment, the invention provides a kit including: (i) a test cell comprising an exogenous nucleic acid sequence encoding a target protein; (ii) a control cell; and (iii) instructions for detection of anti-target antibody in a sample.

In one aspect, the test cell and the control cell are selected from the group consisting of an adherent fixed and permeabilized cell, a suspension of fixed and permeabilized cell and a cell lysate coated on a surface. In another aspect, the kit further includes a detectably labeled anti-Ig antibody. In one aspect, the kit further includes an anti-lysosome marker antibody. In another aspect, the kit further includes an anti-Golgi marker antibody. In one aspect, the cells are adhered to a solid support. In one aspect, the target protein is a SARS-CoV-2 protein.

### BRIEF DESCRIPTION OF THE DRAWINGS

FIG. 1 illustrates an exemplary high copy replicating vector.

FIG. 2 illustrates an exemplary integrating transposon.

FIG. 3 shows the detection by immunofluorescence of anti SARS-CoV-2 S antibody in a sample using 293 cells expressing a pCG115 vector, and as compared to control non-transfected 293 cells.

FIG. 4 shows the detection by immunofluorescence of anti SARS-CoV-2 N antibody in a sample using 293 cells expressing a pCG110 vector, and as compared to control non-transfected 293 cells.

FIG. 5 shows the detection by immunofluorescence of anti SARS-CoV-2 M antibody in a sample using 293 cells expressing a pCG114 vector, and as compared to control non-transfected 293 cells.

FIG. 6 shows the detection by immunofluorescence of anti SARS-CoV-2 E antibody in a sample using 293 cells expressing a pCG112 vector, and as compared to control non-transfected 293 cells.

FIG. 7 shows immunoblots illustrating the expression of the SARS-CoV-2 N, S**, and M proteins in Htet1/N, Htet1/S** and Htet1/M cell lines in the presence or absence of doxycycline. MW markers, from top, 250 kDa, 150 kDa, 100 kDa, 75 kDa, 50 kDa, 37 kDa, 25 kDa, 20 kDa, 15 kDa, 10 kDa.

FIG. 8 is a bar graph illustrating of COVID-19 patient plasma antibody levels to S1 as determined by ELISA, showing average (bar height), standard error of the mean (error bars), and data from individual trials (triangles).

### DETAILED DESCRIPTION OF THE INVENTION

The present invention is based on the seminal discovery that the presence of a target specific antibody in a sample can be detected using a test cell including exogenous nucleic acid sequences encoding one or more target proteins.

Before the present compositions and methods are described, it is to be understood that this invention is not limited to particular compositions, methods, experimental conditions described; as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Nucleic Acids

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e. transfection of, cells, in particular, in the form of RNA which can be prepared by in vitro transcription from a DNA template. The RNA can moreover be modified before application by stabilizing sequences, capping, and polyadenylation.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. 7,957,913; U.S. Pat. 7,776,616; U.S. Pat. 5,234,809; U.S. Pub. 2010/0285578; and U.S. Pub. 2002/0190663.

In one embodiment, the invention provides an isolated nucleic acid sequence encoding one or more SARS-CoV-2 structural proteins selected from S, N, M, E or any combination thereof.

Isolated nucleic acid sequences, or alternatively, “codon-optimized” sequences of nucleic acid sequences of interest, modified to provide the sequences with preferred optimized characteristics can be provided. Such characteristics may include, transcription, translation, post-translational modification, stability of the encoded protein, etc.

A SARS-CoV-2 virion is approximately 50-200 nanometers in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the complete viral envelope, which are the proteins of interest in regard to the present invention. The spike protein, S, which has been imaged at the atomic level using cryogenic electron microscopy, is the protein responsible for allowing the virus to attach to and fuse with the membrane of a host cell. As used herein, the phrase SARS-CoV-2 structural “protein S, N, M, and/or E” refers to the spike (S), nucleocapsid (N), membrane (M), and/or envelope (E) proteins, respectively, which are encoded by the nucleic acid sequences of the invention, or by a codon-optimized oligonucleotide sequence, encoding each protein individually, or any combination of 2 or 3 proteins, or a combination of all 4 proteins. When two or more nucleic acid sequences are included in a single vector or construct, they are in operable linkage such that the each of the 2, 3, or 4 SARS-CoV-2 structural proteins are properly encoded and expressed.

Nucleic acid sequences encoding additional SARS-CoV-2 proteins, such as orfa or orfa/b polypeptides are also included in the nucleic acid sequences of the present invention. Such nucleic acid sequences may be incorporated in a vector as described herein to provide a variation of these vectors. Cells transfected with a vector as described herein, may be transfected with a vector including a nucleic acid sequence encoding an additional SARS-CoV-2 protein and may be used to prepare lysates, plates, assays kits for use in the methods described herein.

### Expression Vectors

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as one or more genes encoding one or more proteins of interest, one or more protein tags, functional domains and the like. In a specific embodiment the proteins of the present invention include SARS-CoV-2 structural protein S, N, M, and/or E.

The expression vector of the invention can include regulatory elements controlling transcription generally derived from mammalian, microbial, viral or insect genes, such as an origin of replication to confer the vector the ability to replicate in a host, and a selection gene to facilitate recognition of transformants may additionally be incorporated. Those of skill in the art can select a suitable regulatory region to be included in such a vector depending on the host cell used to express the gene(s).

For example, the expression vector usually comprises one or more promoters, operably linked to the nucleic acid of interest, capable of facilitating transcription of genes in operable linkage with the promoter. Several types of promoters are well known in the art and suitable for use with the present invention. The promoter can be constitutive or inducible.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include doxycycline, puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyltransferase (XGPRT). Such markers are useful for selecting stable transformants in culture.

In another embodiment, the invention provides a vector including an isolated nucleic acid sequence encoding SARS-CoV-2 structural proteins selected from S, N, M, E or any combination thereof, in operable linkage.

In one aspect, the vector includes nucleic acid sequences encoding SARS-CoV-2 structural proteins S, N, M, E, or any combination thereof wherein the nucleic acid sequences are operably linked in any order.

The vector of the invention can for example include nucleic acid sequences encoding 1, 2, 3, or the 4 SARS-CoV-2 structural proteins.

In other aspects, the vector is a high copy replicating vector or an integrating transposon.

Other non-limiting examples of vectors, suitable for use for the expression of high levels of recombinant proteins of interest include those selected from baculovirus, phage, plasmid, phagemid, cosmid, fosmid, bacterial artificial chromosome, viral DNA, Pl-based artificial chromosome, yeast plasmid, transposon, and yeast artificial chromosome. For example, the viral DNA vector can be selected from vaccinia, adenovirus, foul pox virus, pseudorabies and a derivative of SV40. Suitable bacterial vectors for use in practice of the invention methods include pQE70TM, pQE60TM, pQE-9TM, pBLUESCRIPTTM SK, pBLUESCRIPTTM KS, pTRC99aTM, pKK223-3TM, pDR540TM, PACTM and pRIT2TTM. Suitable eukaryotic vectors for use in practice of the invention methods include pWLNEOTM, pXTITM, pSG5TM, pSVK3TM, pBPVTM, pMSGTM, and pSVLSV40TM. Suitable eukaryotic vectors for use in practice of the invention methods include pWLNEOTM, pXTITM, pSG5TM, pSVK3TM, pBPVTM, pMSGTM, and pSVLSV40TM. One type of vector is a genomic integrated vector, or “integrated vector,” which can become integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

### Cells

In one embodiment, the invention provides an isolated cell, e.g., mammalian cell, including a vector including an isolated nucleic acid sequence encoding SARS-CoV-2 structural proteins selected from S, N, M, E or any combination thereof, in operable linkage.

The nucleic acid construct (or the vector) of the present invention may be introduced into a host cell to be altered thus allowing expression of the protein within the cell. A variety of host cells are known in the art and suitable for proteins expression and extracellular vesicles production. Examples of typical cell used for transfection include, but are not limited to, a bacterial cell, a eukaryotic cell, a yeast cell, an insect cell, or a plant cell. For example, Human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F.

The nucleic acid construct of the present invention, included into a vector, may be introduced into a cell to be altered thus allowing expression of the chimeric protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE ™ DOJINDO HILYMAX ™ FUGENE ™ JETPEI ™ EFFECTENE ™ and DREAMFECT ™.

In one aspect, the cell expresses at least one SARS-CoV-2 structural protein selected from S, N, M or E.

In one embodiment, the invention provides a method of detecting the presence of a target specific antibody in a sample including: (i) contacting the sample with a support containing a test cell including one or more exogenous nucleic acid sequences encoding one or more target proteins; and (ii) detecting the presence of the target specific antibody in the sample by contacting the immune complex of (i) with an anti-immunoglobulin (Ig) antibody, and detecting the anti-immunoglobulin (Ig) antibody.

A “target” as used herein refers to any polypeptide, or fragment thereof that can constitute an antigen. An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, which results in an immune reaction against the antigen. An antigen is preferably a product which corresponds to or is derived from a naturally occurring antigen. Such antigens include SARS-CoV-2 structural proteins S, N, M, and E, and any variants or mutants thereof.

The term “epitope” refers to an antigenic determinant in a molecule such as an antigen, i.e., to a part in or fragment of the molecule that is recognized by the immune system. An epitope of a protein such as a viral antigen preferably includes a continuous or discontinuous portion of said viral protein, to elicit an immune reaction and the generation and specific antibodies directed against the epitope.

As used herein the terms “antibody” refers to immunoglobulin (Ig) molecules and immunologically active portions of immunoglobulin molecules, i.e., molecules that contain an antigen-binding site that specifically binds an antigen. “Native antibodies” and “intact immunoglobulins”, or the like, are usually heterotetrameric glycoproteins of about 150,000 Daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and µ, respectively. The subunit structures and three-dimensional configurations of different classes of immunoglobulins are well known.

The antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells).

A “neutralizing antibody”, or “Nab” is an antibody that defends a cell from a pathogen or infectious particle by neutralizing any effect it has biologically. Neutralization renders the particle no longer infectious or pathogenic. Neutralizing antibodies are part of the humoral response of the adaptive immune system against viruses, intracellular bacteria and microbial toxin. By binding specifically to surface antigen on an infectious particle, neutralizing antibodies prevent the particle from interacting with its host cells it might infect and destroy. Immunity due to neutralizing antibodies is also known as sterilizing immunity, as the immune system eliminated the infectious particle before any infection took place.

By “detecting the presence” of an antibody in a sample, is it meant that the methods described herein can be used for identifying samples that contain one or more antibodies, regardless of the type, class, and activity; as long as the antibody, or the fragment thereof, specifically binds to a pathogen specific antigen. Using additional means, such as standard curves for example, the methods described herein can be used to evaluate a titer of antibody present in a sample.

The term “sample” is meant to refer to any composition potentially comprising an analyte, e.g., an antibody. A “biological sample” is meant to refer to any “biological specimen” collected from a subject, and that is representative of the content or composition of the source of the sample, considered in its entirety. A sample can be collected and processed directly for analysis, or be stored under proper storage conditions to maintain sample quality until analyses are completed. Ideally, a stored sample remains equivalent to a freshly collected specimen. The source of the sample can be an internal organ, vein, artery, or even a fluid. Non-limiting examples of sample include blood, plasma, urine, saliva, sweat, organ biopsy, cerebrospinal fluid (CSF), or tears. Specifically, the present invention relies on the use of any biological fluid collected from a subject that can contain antibody.

The sample can be used directly upon collection, or be stored appropriately before being used (e.g., refrigerated, frozen, etc.). The sample can be used directly, or be prepared to be more suitable to the methods (e.g., the sample can be diluted, concentrated, or mixed with preservatives, any other method of preparation of the sample can be implemented).

The term “subject” as used herein refers to any individual or patient to which the subject methods are performed. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., non-human primate and primates (including monkeys, chimpanzees, orangutans and gorillas) are included within the definition of subject.

In one aspect, the sample is further contacted with a control cell, wherein a target specific antibody present in the sample forms an immune complex with the one or more pathogen proteins expressed by the test cell.

The sample can for example be contacted with a test cell, or with a cell mixture including both a test cell and a control cell. In some aspects, the sample is contacted with a support containing a test cell and a control cell.

As used herein, the term “test cell” refers to a cell that has been modified to express one or more pathogen proteins, and that can be used to evaluate the presence of an antibody in a sample. Because a test cell expresses pathogen proteins, immune complexes can be form in a test cell between a pathogen protein and a pathogen specific antibody contacted therewith, and present in a sample. The term “immune complex” refers to a molecule formed from the binding of multiple antigens to antibodies. Also referred to as an antigen-antibody complex or antigen-bound antibody, the immunes complexes described in the present invention can include any complex formed between a pathogen protein (or any fragment, variant or mutant thereof) and an antibody or binding fragment thereof capable of specifically binding to a pathogen epitope. Immunes complexes, as any protein complexes can in turn be recognized by specific antibodies, such as anti-immunoglobulin antibodies, that specifically recognize and bind to Ig.

A “control cell”, which may be transformed to express one or more exogenous nucleic acid sequences, does not include a nucleic acid sequence encoding a pathogen protein. A control cell constitutes an internal negative control to the method, to ascertain the accuracy of the results.

The control cell serves as an internal control, and therefore needs only be present in limited amount (e.g., in an amount that is less that the amount of test cell). In one aspect, the test cell and control cell are present in a ratio of about 9:1 to 4:1.

There are various ways to detect the presence of a specific antibody in a sample. Antibodies being large proteins, they can be detected using antibodies that specifically recognize antibodies (or immunoglobulins). For example, an anti-immunoglobulin antibody may bind to either the constant region or to the variable region of an antibody. The target specific antibody described herein, which form complexes with target proteins expressed in the test cells may be detected by detecting immunocomplexes including a target protein and an anti-target protein specific antibody.

In one aspect, detecting the presence of the target specific antibody includes contacting the immune complex with an anti-immunoglobulin (Ig) antibody.

The test cell can include exogenous nucleic acid sequences encoding one or more target proteins. In one aspect, the target protein is a pathogen protein.

A pathogen, which can be a bacteria, virus, or any other microorganism that can cause a disease in a subject, can elicit an immune response (i.e., an integrated bodily response to a pathogen antigen, which can include a cellular immune response and/or a humoral immune response) in the subject. For example, upon contact and/or exposure to a pathogen, a subject may respond with an humoral immune response, characterized by the production of antibody, specifically directed against one or more pathogen antigens.

In one aspect, the pathogen is a virus or a bacteria. In an illustrative example of the invention, the pathogen is SARS-CoV-2 virus.

Because of the very high replication rate of virus such as SARS-CoV-2, ~10¹⁰ new virions are produced per day; given the high error rate of virion reverse transcriptase (approximately 1 base error per 10⁴ nucleotides transcribed), the emergence of mutations and their rate is high. The pathogen protein used in the methods described herein can therefore be a wild-type (WT) pathogen protein (corresponding to the polypeptide sequence of the protein when it was first established), or a mutant pathogen protein. Mutant pathogen protein can be isolated from newly emerged virus, or be the result of empiric mutagenesis to generate multiple protein variants.

Various variants and/or mutants of SARS-CoV-2 proteins can be generated, based on the naturally occurring variants of the virus, or by molecular engineering. For example, several variants of SARS-CoV-2 S protein are described, and may have different behaviors upon expression in a cell. The S** variant primarily localizes at the plasma membrane. S^{W1} protein and S^{D614G} protein include a mutation affecting the subcellular localization of the protein, which induces protein retention in the Golgi apparatus, or in lysosomes.

In some aspects, the SARS-CoV-2 S protein is an S^{W1} protein, an S^{D614G} protein or a S** protein.

In one aspect, the test cell and the control cell are available for the assay as an adherent fixed and permeabilized cell, a suspension of fixed and permeabilized cells, or a cell lysate coated on a surface of a support. Following contact with a sample and an Ig molecule to detect the presence of antibody in the sample, the immune complex is detected, for example, by immunofluorescent microscopy, flow cytometry, or enzyme-linked immunosorbent assay (ELISA).

There are various way to prepare stable forms of the cells (test cells and control cells) For example, the cells can be on or in a support capable of receiving the sample. For example, the support can be a test tube, a well plate (e.g., 96 well plate), a tissue culture plate or dish, a cover glass, or any other support that allow cells to be fixed in some manner prior to be contacted with the sample.

Stable forms of the test cells and control cells can be an adherent cell, fixed and permeabilized for example. The cell can be cultured in a 96-well plate or on a cover glass or equivalent material, for example, and fixed and permeabilized when the appropriate confluence is reached. Using fluorescently labeled anti Ig antibodies as secondary antibodies in a classic immunoassay, where the primary antibodies are the pathogen-specific antibodies present in the biological sample, the immune-complexes is detected by immunofluorescent microscopy.

Stable forms of the test cells and control cells can be a suspension of fixed and permeabilized cell. The cells can be cultured until the required amount of cells is reached, the cells can then be collected in a suspension, fixed and permeabilized. Using fluorescently labeled anti Ig antibodies as secondary antibodies in a classic immune assay, where the primary antibodies would be the pathogen-specific antibody present in the biological sample, the immune-complexes can be detected by detecting of the fluorescence by flow cytometry.

Alternatively, the stable form of the test cells and control cells can be a cell lysate coated on a surface. The cells can be cultured until a sufficient amount of cells to prepare cell lysate is reached, the cells can then be collected in a suspension, and cell lysates prepared. The cell lysate can be coated in EIA, ELISPOT plates, or any other plate suitable for such assay, such as any flat bottom clear, polystyrene plate having a high binding surface capability. Using AP/HRP-tagged anti Ig antibodies as secondary antibodies in a classic EIA assay, or ELISPOT, where the primary antibodies would be the pathogen-specific antibody present in the biological sample, the immune-complexes can be detected.

There are various ways to fix test and control cells to preserve and stabilize cell morphology; to inactivate proteolytic enzymes that could otherwise degrade the sample; to strengthen samples so that they can withstand further processing and staining; and to ensure that the antigenic sites remain accessible to the detection reagents being used. Non-limiting examples of fixative agents that can be used to fix cells include: 4% (w/v) Paraformaldehyde, 4% (w/v) Paraformaldehyde-1% (v/v), glutaraldehyde, 10% Neutral-buffered formalin (NBF), Bouin’s fixative, Zenker’s solution, Helly solution, Carnoy’s solution, ice-cold acetone (100%) or methanol (100%), and 1% (w/v) osmium tetroxide. The choice of fixative and fixation protocol may depend on the additional processing steps and final analyses that are planned.

In addition to fixation, the cells can be permeabilized, to generate pores on the cell membrane, to allow the detection of intracellular antigens with a primary antibody because it allows entry through the cell membrane. Permeabilization is generally introduced after cells have been prepared with a fixative agent to initiate protein cross-linking. The two most common agents used to permeabilize the cell membrane are the detergents Triton-X 100 or Tween-20, with Tween-20 being the more gentle of the two. Triton-X 100 inserts a detergent monomer into the lipid membrane ultimately permeabilizing the membrane, whereas Tween-20 has a more renaturing effect on proteins and might improve antibody-antigen binding. Typically, the permeabilization agents are diluted into a phosphate buffer solution (PBS) in order to create enough volume required to incubate the entire sample. Determining which permeabilization agent to use and the amount of exposure to a permeabilization agent is dependent on the sample, and can also be adjusted depending on the additional processing steps and final analyses that are planned.

Any additional assays capable of detection immune-complexes can be used in the methods of the invention.

In various aspects, the anti Ig antibody detects an immune-complex including a target protein expressed by the test cell and an anti-target specific antibody present in the sample.

In one aspect, detecting an anti-target specific antibody in the sample includes detecting the detectably labeled anti-Ig antibody and the detectable protein in the test cell but not in the control cell.

In one aspect, the anti-Ig antibody is an anti IgG, IgM, or IgA antibody, or a combination thereof.

In one aspect, the anti IgG, IgM, or IgA antibody is detectably labeled.

By detectably labeled, it is meant that the anti-Ig antibody includes a tag to allow for the detection of the antibody. The anti IgG, IgM, or IgA antibodies can be labeled in various ways; non-limiting examples include fluorescent labeling such as using GFP, YFP, EGFP, FITC, ALEXA FLUOR™, Cy5, AMCA, Cy2, fluorescein, rhodamine (TRITC), R-Phycoerythrin (RPE), ATTOs, TEXAS RED ™ allophycocyanin, and DYLIGHT ™. The antibodies can also be labeled with enzymes, for enzymatic-based immunocomplexes detection. Examples of enzymes include alkaline phosphatase (AP) and horseradish peroxidase (HRP).

In one aspect, the anti-pathogen specific antibody is an anti-SARS-CoV-2 antibody selected from an anti- S protein antibody, an anti- N protein antibody, an anti- M protein antibody, an anti- E protein antibody or a combination thereof.

The methods described herein allow for the successive or concurrent analysis of a same sample for the detection of more than one pathogen specific antibody. For example, a sample can be tested for detecting the presence of anti-SARS-CoV-2 antibodies. The anti-SARS-CoV-2 antibody can be an anti- S protein antibody, an anti- N protein antibody, an anti-M protein antibody, an anti- E protein antibody or a combination thereof. Accordingly, a sample can be found to include more than one pathogen specific antibodies, which can provide a multiplex analysis, allowing to ascertain the results obtained.

In some aspects, the anti-pathogen specific antibody detected is an anti-S protein antibody. In other aspects, an anti-N antibody is further detected with the anti-S protein antibody. In another aspect, an anti-M antibody is further detected.

For example, the anti-pathogen specific antibodies detected are an anti-S protein antibody and an anti-N antibody; or an anti-S protein antibody, an anti-N antibody and an anti-M antibody.

The detection of an anti-pathogen specific antibody in a sample collected from a subject indicates that immune cells of the subject have been presented with the pathogen, or any antigen derived therefrom; and that an immune reaction took place in the subject, leading to the production of antibodies directed against the pathogen. The detection of antibodies in a sample collected from a subject can indicate the presence of innate antibodies, present in the subject while the contact with the pathogen is on-going, and the presence of memory antibodies, that can be detected in a subject even after extended period of time after being contacted with the pathogen. In one aspect, detecting an anti-pathogen specific antibody in the sample indicates that the subject is infected by the pathogen, has developed an immunity against a pathogen related disease and/or infection, and/or is vaccinated against the pathogen related disease and/or infection.

In another aspect, detecting the detectably labeled anti-Ig antibody alone in a test cell, detecting the detectable protein alone in the test cell, or detecting the detectably labeled anti-Ig antibody and/or the detectable protein in a control cell indicates an absence of anti-pathogen specific antibody in the sample.

The methods of the invention rely on the contacting a biological fluid collected from a subject, which contains antibody with a test cell expressing high levels pathogen protein, and a control cell that do not express pathogen proteins. Any antibody present in the sample that is directed to an epitope of a pathogen protein can recognize and bind to the epitope. The resulting complex (or immune complex) is then contacted with antibodies directed against immunoglobulins, to allow the detection of any pathogen specific antibody present in the sample and bound to the epitope presented in the test cell.

By detecting, for example, the presence of anti SARS-CoV-2 antibodies in a sample collected from a subject, the method can identify and differentiate subjects who do or do not have an immune response to SARS-CoV-2.

By providing information regarding the type of antibody (i.e., IgG, IgM, and/or IgA antibodies) and the target of the antibody (i.e., anti-protein S, anti-protein N, anti-protein M and/or anti-protein E) produced by a subject in response to a SARS-CoV-2 infection), the method also provides the ability to characterize the immune reactions of people infected with SARS-CoV-2, and to identify which viral protein is the most important to produce a robust antibody production in response to the infection.

The methods described herein represent a ground-breaking improvement, as they allow for the analysis of more complex immune reactions that possible using assays based on single proteins or viral proteins expressed outside the context of a human cell.

Proteins can be subjected to mutations in their polypeptide sequence, which can affect among other the subcellular localization of protein in the cell, by modifying the retention of the protein in a cellular organelle. The methods described herein allow for the detection of anti-target specific antibodies in cells. The co-detection of proteins that are specifically expressed in certain cellular organelle can be used to specify where in the cell (e.g., in which subcellular compartment, and in which cellular organelle) a protein target is expressed, and therefore antibodies against which variant or mutant of a target protein are present in the sample.

In one aspect, a sub-cellular localization of the target specific antibody is further detected. In one aspect, detecting the subcellular localization of the target specific antibody includes contacting the cells with one or more organelle-specific antibodies and determining co-localization of the target specific antibody and the organelle-specific antibodies.

As used herein, the term “organelle” refers to a specialized subunit, usually within a cell, that has a specific function. Organelles are either separately enclosed within their own lipid bilayers (also called membrane-bound organelles) or are spatially distinct functional units without a surrounding lipid bilayer (non-membrane bound organelles). Non-limiting example of organelle include: nucleus, mitochondria, nucleoli, ribosome, endoplasmic reticulum (ER), Golgi apparatus, vacuole, and lysosome.

In one aspect, detecting the presence of the target specific antibody includes detecting the target specific antibody directly or indirectly by immunofluorescence microscopy.

The methods described herein can be applied using more than one test cells, or test cells that express more than one target protein, such that anti-target specific antibodies directed against more than one epitope of a target can be detected. For example, mutants or variants or a target protein can be detected along with a wild-type target protein. The co-detection of subcellular organelles can then provide information related to both the presence of the anti-target specific antibodies in a sample, and the presence of anti-target specific antibodies recognizing variants or mutants of the target protein. In cases where the target protein is a pathogen protein, such as a viral protein, it can indicate that the subject, from which a sample has been collected, has been contacted with a variant or a mutant of the pathogen or virus.

In other aspects, a subcellular localization of a target specific antibody is further detected in a second test cell, wherein the target protein in a first test cell and in the second test cell localize differently.

In one aspect, the first and second target proteins are mutant variants of one another. In other aspects, the sample is collected from a subject, the target protein is SARS-CoV-2 Spike protein a SW1 protein, a SD614G protein or a S** protein, and detecting sub-cellular localization of the target specific antibody includes the detection of localization to the Golgi, wherein localization to the Golgi indicates the presence of SARS-CoV-2 neutralizing antibodies in the sample. In another aspect, the target specific antibody is detectably labeled.

In some aspects, the one or more organelle-specific antibodies is an anti-lysosome marker antibody, an anti-Golgi marker antibody, an anti-ER-Golgi-intermediate compartment antibody, a plasma membrane antibody, or a combination thereof.

In some aspects, the one or more organelle-specific antibodies is an anti-lysosome marker antibody. In another aspect, the cells are further contacted with an anti-Golgi marker antibody.

By further contacting the cells with an anti-lysosome marker antibody and/or with an anti-Golgi marker antibody, subcellular compartments can be visualized and/or detected, and subcellular localization of the pathogen proteins can evaluated (by detecting co-localization of the antibody and the subcellular compartment marker). Modifications of proteins structural conformation, such as those resulting from mutations can affect protein trafficking in the cells, and therefore have an impact on subcellular retention of proteins. For example, pathogen protein mutations (e.g., resulting from a mutations in the pathogen genome) can alter the protein conformation, which can result in protein retention in the Golgi, or in lysosomes. Combining the use of test cell expressing variants and/or mutants of pathogen protein, with additional staining allowing the determination of subcellular localization can thus be useful. Determining the subcellular distribution of the anti-pathogen specific antibodies can therefore provide additional information regarding the structural conformation of the pathogen protein recognized by the pathogen-specific antibody, and regarding which potential alternative pathogen variants the subject has been infected with. Such information may be informative as to the pathogenicity of the pathogen, in cases where specific conformational variants are specifically associated with certain pathogen characteristics (virulence, replication, titer, etc).

Lysosomes are membrane-bound organelles found in many animal cells. They are spherical vesicles that contain hydrolytic enzymes that can break down many kinds of biomolecules. A lysosome has a specific composition, of both its membrane proteins, and its lumenal proteins. The lumen’s pH (~4.5-5.0) is optimal for the enzymes involved in hydrolysis, analogous to the activity of the stomach. Besides degradation of polymers, the lysosome is involved in various cell processes, including secretion, plasma membrane repair, apoptosis, cell signaling, and energy metabolism. Lysosomes act as the waste disposal system of the cell by digesting in use materials in the cytoplasm, from both inside and outside the cell. Material from outside the cell is taken-up through endocytosis, while material from the inside of the cell is digested through autophagy.

LAMP1 and LAMP2 glycoproteins comprise about 50% of all lysosomal membrane proteins lysosomal-associated membrane protein (LAMP), such as LAMP1 and LAMP2, which are thought to be responsible in part for maintaining lysosomal integrity, pH and catabolism.

Lysosomal-associated membrane protein 1 (LAMP-1) also known as lysosome-associated membrane glycoprotein 1 and CD107a (Cluster of Differentiation 107a), is a protein that in humans is encoded by the LAMP1 gene. LAMP 1 resides primarily across lysosomal membranes, and functions to provide selectins with carbohydrate ligands. CD107a has also been shown to be a marker of degranulation on lymphocytes such as CD8+ and NK cells and may also play a role in tumor cell differentiation and metastasis.

The mammalian target of rapamycin (mTOR), is a kinase that in humans is encoded by the MTOR gene. mTOR is a member of the phosphatidylinositol 3-kinase-related kinase family of protein kinases, and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. As a core component of mTORC2, mTOR also functions as a tyrosine protein kinase that promotes the activation of insulin receptors and insulin-like growth factor 1 receptors. mTORC2 has also been implicated in the control and maintenance of the actin cytoskeleton. mTOR is the catalytic subunit of two structurally distinct complexes: mTORC1 and mTORC2. Both complexes localize to different subcellular compartments, thus affecting their activation and function. Upon activation by Rheb, mTORC1 localizes to the Ragulator-Rag complex on the lysosome surface where it then becomes active in the presence of sufficient amino acids.

In some aspects, the anti-lysosome marker antibody binds to a marker selected from the group consisting of Lamp1, Lamp2, CD63/Lamp3 and mTOR.

In other aspects, the sample is a plasma sample and the ratio of the plasma sample to the anti-lysosome antibody is about 1:50 to 1:100,000; 1:100 to 1:50,000; 1:500 to 1:25,000; 1:500 to 1:10,000. In one aspect, the ratio is about 1:1000 to 1:5000.

The Golgi is an organelle found in most eukaryotic cells. Part of the endomembrane system in the cytoplasm, it packages proteins into membrane-bound vesicles inside the cell before the vesicles are sent to their destination. It resides at the intersection of the secretory, lysosomal, and endocytic pathways. It is of particular importance in processing proteins for secretion, containing a set of glycosylation enzymes that attach various sugar monomers to proteins as the proteins move through the apparatus. The Golgi apparatus is a major collection and dispatch station of protein products received from the endoplasmic reticulum (ER). Proteins synthesized in the ER are packaged into vesicles, which then fuse with the Golgi apparatus. These cargo proteins are modified and destined for secretion via exocytosis or for use in the cell. In this respect, the Golgi can be thought of as similar to a post office: it packages and labels items which it then sends to different parts of the cell or to the extracellular space. The Golgi apparatus is also involved in lipid transport and lysosome formation.

Golgi subfamily A member 2 (GOLGA2, or GM130) is a protein that in humans is encoded by the GOLGA2 gene. The Golgi apparatus, which participates in glycosylation and transport of proteins and lipids in the secretory pathway, consists of a series of stacked cisternae (flattened membrane sacs). The golgins are a family of proteins, of which the protein encoded by this gene is a member, that are localized to the Golgi. This encoded protein has been postulated to play roles in the stacking of Golgi cisternae and in vesicular transport. Several alternatively spliced transcript variants of this gene have been described, but the full-length nature of these variants has not been determined.

In one aspect, the Golgi marker is GM130.

In another embodiment, the invention provides an isolated peptide

DSEPVLKGVKLHYT (SEQ ID NO: 1).

The terms “peptide”, “polypeptide” and “protein” are used interchangeably herein and refer to any chain of at least two amino acids, linked by a covalent chemical bound. As used herein peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In some aspects, an isolated peptides can be used to immunize a subject. By immunization, it is meant that the peptide can generate an immune reaction in the subject, and induce, for example, the production by the subject of antibodies specifically directed against the peptide. Such antibodies can bind the peptide with high specificity and sensitivity.

In an additional embodiment, the invention provides an antibody that specifically binds to the peptide of SEQ ID NO: 1.

In one embodiment, the invention provides a kit including: (i) a test cell comprising an exogenous nucleic acid sequence encoding a target protein; (ii) a control cell; and (iii) instructions for detection of anti-target antibody in a sample.

In one aspect, the test cell and the control cell are selected from the group consisting of an adherent fixed and permeabilized cell, a suspension of fixed and permeabilized cell and a cell lysate coated on a surface. In one aspect, the target protein is a SARS-CoV-2 protein.

The present invention relates to mammalian cells transfected with expression vectors to express high levels of SARS-CoV-2 S, N, M, and E proteins all together, singly, or in any combination, the SARS-CoV-2 protein encoded can be WT proteins, or any mutant or variant thereof. Kits of the present invention can include such cell in any shape or form, as long as it allows for the intended uses described herein (i.e., detecting and/or quantifying anti SARS-CoV-2 antibodies in a sample). Non limiting examples of shape/and or form of cell that can be included in the kit include: assay plates carrying these mammalian cells in fixed, fixed & and permeabilized, or lysed forms; lysates of these mammalian cells, generated in ways known to preserve native protein conformation and assembly context of the virus, in ways known to denature proteins, or some combination of the two; assay plates coated with these lysates for the purpose of detecting, measuring, and characterizing antibody responses of subjects that have or have not been infected with SARS-CoV-2, immunized with vaccines directed against antigens encoded by SARS-CoV-2, other related viruses, or any agent; and cells expressing the SARS-CoV-2 S, N, M, or E proteins, either singly or in combination.

Any variations of these cells, lysates, plates and assays that include additional SARS-CoV-2-encoded proteins, in particular the orfa or orfa/b polypeptides are included in the present disclosure and are part of the present invention.

In another aspect, the kit further includes a detectably labeled anti-Ig antibody.

In one aspect, the kit further includes an anti-lysosome marker antibody.

In another aspect, the kit further includes an anti-Golgi marker antibody.

In one aspect, the cells are adhered to a solid support.

The kit may also include any reagents necessary for the realization of the assays.

“Instructions” are used herein may include protocols, know-how, best practices related to measuring the presence of antibodies, and to analyze the results.

Presented below are examples discussing the vector encoding SARS-CoV-2 structural proteins S, N, M, and/or E, recombinant cell including the vectors, small extracellular vesicle loaded with SARS-CoV-2 structural protein S, N, M, and/or E including vaccine composition, and methods and kits of use thereof, contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Vectors and Cell Genration

To generate recombinant cells producing high levels of the vector SARS-CoV-2 structural protein S, N, M, and/or E, several expression vectors were synthesized.

SARS-CoV-2 genes ere synthesized as codon optimized ORFs, and were then clones as untagged or tagged (p2a tag) ORFs.

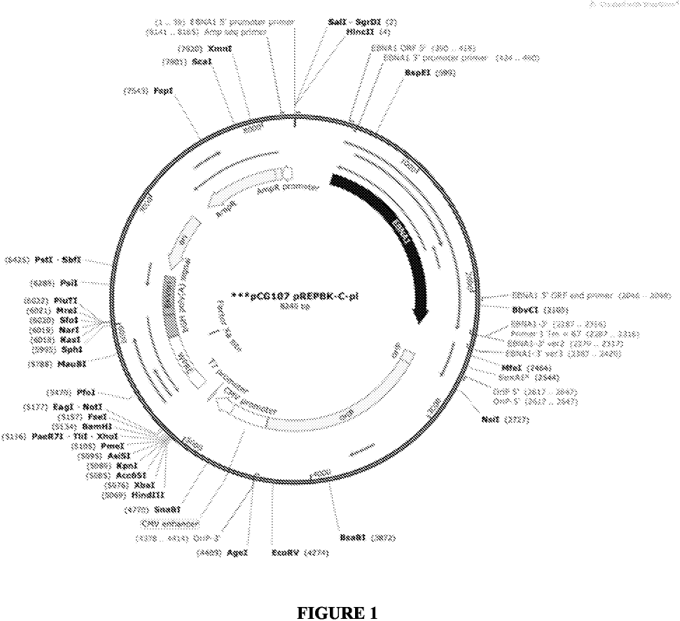

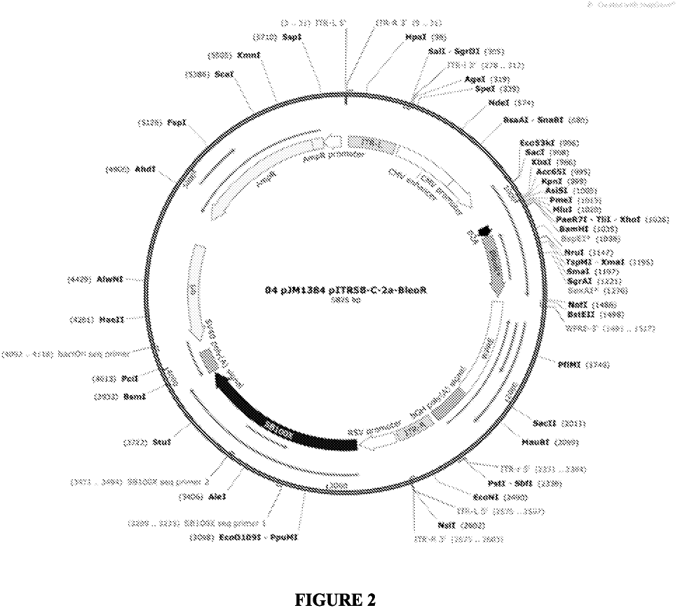

As illustrated in FIGS. 1 and 2, the codon-optimized ORFs were either cloned into high copy replicating vector (FIG. 1) or into integrating transposon (FIG. 2).

High copy replicating vector and integrating transposon including either 1, 2, 3, or the 4 SARS-CoV-2 structural proteins were generated, and transfected into host cells to obtain either cells expressing individually only one structural protein, or cells expressing 2, 3 or the 4 structural proteins of SARS-CoV-2 in one cell. Alternatively, host cells were transfected with multiple vectors encoding 1, 2, or 3 structural proteins, so that the combination of the vectors transfected lead to the expression of several proteins in a same cell.

### Example 2

### Detection of Antibody Anti Sars-cov-2 S/n/m/e Poteins in Cells by Ifm

To measure antibody presence, titer, type, and target in response to a SARS-CoV-2 infection or a SARS-CoV-2 vaccination, the presence of anti-S/N/M/E antibodies using Human embryonic kidney 293 (HEK293) cells expressing SARS-CoV-2 S/N/M/E proteins was detected using immunofluorescence microscopy (IFM).

To assess if the detection of SARS-CoV-2 S/N/M/E proteins could be evaluated by IFM, HEK293 cells were cultured and transfected to express high levels of the four structural proteins (S, N, M, and E) individually, each protein being expressed separately from the other in a cell line. A stable form of the cells, in the form of adherent cells cultured on coverglasses, fixed and permeabilized was prepared.

This material was used for measuring anti-SARS-CoV-2 antibodies in human plasma and saliva. After incubation of the human sample (plasma or saliva) with the adherent cells on the coverglasses, the cells were washed, and then incubated with fluorescently-tagged secondary antibodies directed against IgG, IgM, and IgA, in order to detect the immune-complexes formed by antibodies directed against SARS-CoV-2 S/N/M/E proteins present in the sample, and the SARS-CoV-2 S/N/M/E protein expressed in the cell. After an additional wash, fluorescence was detected using an immuno-fluorescent microscope.

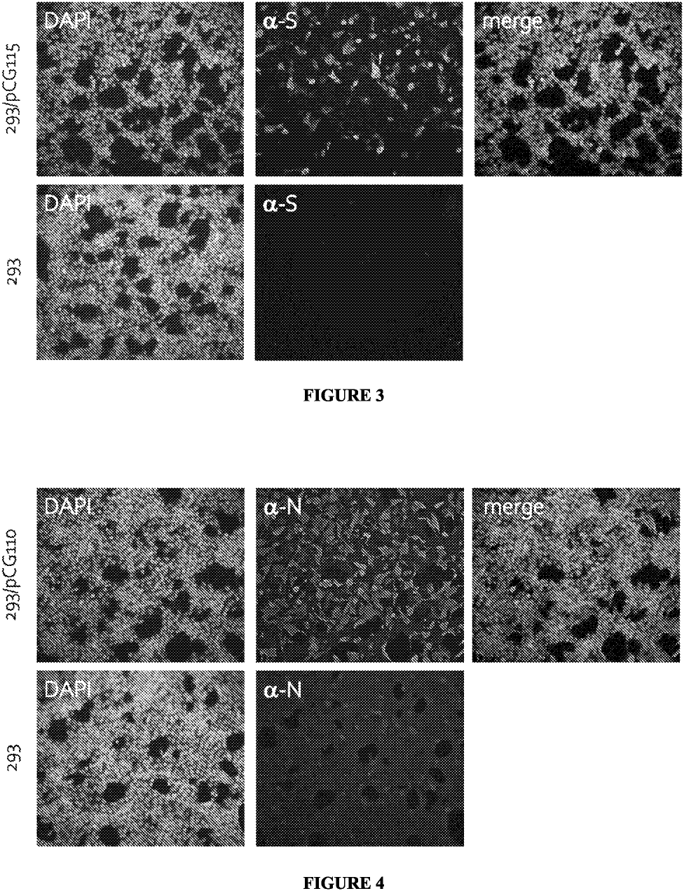

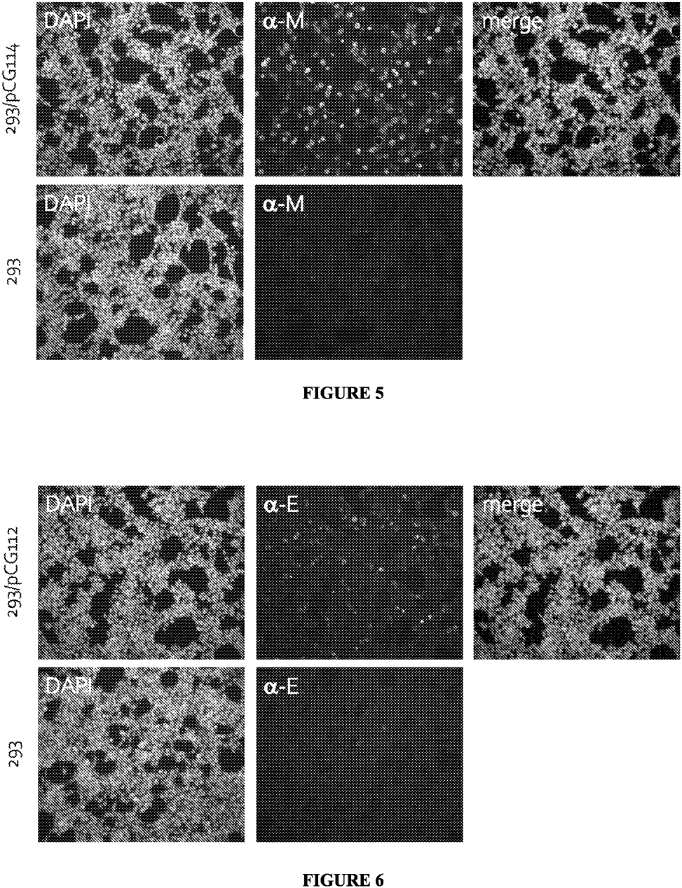

As illustrated in FIGS. 3-6, antibodies against SARS-CoV-2 S protein (FIG. 3), SARS-CoV-2 N protein (FIG. 4), SARS-CoV-2 M protein (FIG. 5), and SARS-CoV-2 E protein (FIG. 6) were detected in the samples by IFM.

### Example 3

### Detection of Antibody Anti Sars-cov-2 S/n/m/e Proteins in Cells by Flow Cytometry

To measure antibody presence, titer, type, and target in response to a SARS-CoV-2 infection or a SARS-CoV-2 vaccination, the presence of anti-S/N/M/E antibodies using HEK293 cells expressing SARS-CoV-2 S/N/M/E proteins was detected using flow cytometry.

To assess if the detection of SARS-CoV-2 S/N/M/E proteins could be evaluated by flow cytometry, HEK293 cells were cultured and transfected to express high levels of the four structural proteins (S, N, M, and E). A stable form of the cells, in the form of a cell suspension of fixed and permeabilized cells was prepared. This material was used for measuring anti-SARS-CoV-2 antibodies in human plasma and saliva. A suspension of negative control cells fluorescently marked (using a DAPI staining) was generated.

A suspension of cells expressing the S, N, M, and E proteins of SARS-CoV-2 was mix at a 20%:80% ratio with the suspension of negative control cells to generate an assay cell suspension. The assay cell suspension was incubated with the sample (human saliva or plasma), which was followed by a wash step.

After the wash, the cells were incubated with fluorescently-tagged secondary antibodies directed against IgG, IgM, and IgA, in order to detect the immune-complexes formed by such antibodies directed against SARS-CoV-2 S/N/M/E proteins present in the sample, and the SARS-CoV-2 S/N/M/E protein expressed in the cell. The cells were then washed, before being assayed by flow cytometry (for each sample, 2 independent assays were prepared).

Antibodies against SARS-CoV-2 S protein, SARS-CoV-2 N protein, SARS-CoV-2 M protein, and SARS-CoV-2 E protein were detected in the sample.

### Example 4

### Detection of Antibody Anti Sars-cov-2 S/n/m/e proteins in Cells by Elisa

To measure antibody presence, titer, type, and target in response to a SARS-CoV-2 infection or a SARS-CoV-2 vaccination, the presence of anti-S/N/M/E antibodies using HEK293 cells expressing SARS-CoV-2 S/N/M/E proteins was detected using ELISA.

To assess if the detection of SARS-CoV-2 S/N/M/E proteins could be evaluated by EIA or ELISPOT assays, HEK293 cells were cultured and transfected to express high levels of the four structural proteins (S, N, M, and E). A stable form of the cells, in the form of a cell lysate of the cultured cells was prepared.

This material was used for measuring anti-SARS-CoV-2 antibodies in human plasma and saliva. After coating the cell lysates on EIA and ELISPOT plates, the sample was incubated on the plates, and then washed. The plates were then incubated with AP/HRP-tagged secondary antibodies directed against IgG, IgM, and IgA in order to detect the immune-complexes formed by such antibodies directed against SARS-CoV-2 S/N/M/E proteins present in the sample, and the SARS-CoV-2 S/N/M/E protein present in the cell lysates.

### Example 5

### Kit for the Detection of Anti-sars-cov-2 Antibodies in a Sample

A kit for the detection of anti SARS-CoV-2 antibody present in a sample will be provided. The kit will include a stable form of a cell expressing high levels of SARS-CoV-2 structural protein S, N, M, and E, which, as described in Examples 2, 3, and 4, can be a coverglass coated with adherent cells, fixed and permeabilized, ready for the realization of an immunofluorescent assay; a suspension of cells fixed and permeabilized, ready for the realization of a flow cytometry analysis; or a EIA/ELISPOT plate, coated with a cell lysate, ready for the realization of an enzyme-based immune detection assay. The kit will be completed by a set of complete instruction for the realization of the assay corresponding to the stable form of the cells (i.e., an immunofluorescence, a flow cytometry analysis, or an EIA/ELISPOT assay). Optionally, the kit will contain all the reagents required for the realization of these assays.

Alternatively, the kit will include a recombinant mammalian cell expressing a vector comprising an expression cassette comprising a codon-optimized oligonucleotide sequence encoding a SARS-CoV-2 structural protein S, N, M, and E, in an expandable form (such as a cryopreserved vial of cells), along with instructions to prepare a stable form of cell therefrom. Such kit will also be completed by a set of complete instruction for the realization of the assay corresponding to the stable form of the cells (i.e., an immunofluorescence, a flow cytometry analysis, or an EIA/ELISPOT assay). Optionally, the kit will contain all the reagents required for the realization of these assays.

### Example 6

### Ifm-based Sars-cov-2 Serology Tests

Most approaches for detecting anti-SARS-CoV-2 antibodies in human biofluids rely on point-of-care devices or ELISA-based laboratory tests. Here, a high-content microscopy-based serology assay that included an in-sample negative control and generated a multidimensional readout of positivity was implemented.

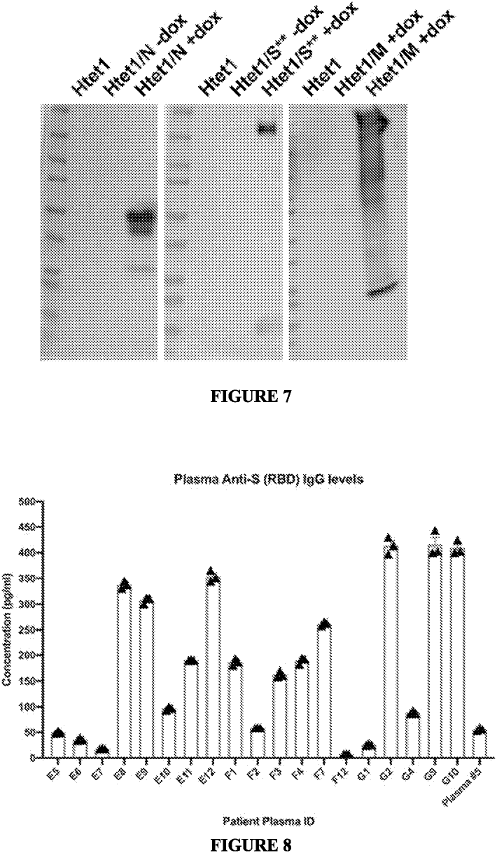

The assay used engineered HEK293 cell lines that encoded for doxycycline-induced expression SARS-CoV-2 structural proteins, including spike, nucleocapsid, and membrane (FIG. 7). Furthermore, the form of spike expressed in the test lines, S**, incorporated multiple mutations known to stabilize spike in a trimeric, prefusion conformational state of the spike protein, a conformation that has been proposed to be superior for the detection of neutralizing antibodies. These included a pair of proline substitutions (986KV987 to 986PP987) and a quartet of amino acid changes that eliminate the S1/S2 cleavage site (682RRAR685 to 682GSAG685). Htetl cells, and Htet1/N, Htet1/S**, and Htet1/M cells were grown in the absence or presence of doxycycline, were lysed and processed for immunoblot using rabbit polyclonal anti-peptide antibodies specific for SARS-CoV-2 (FIG. 7, left panel) nucleocapsid protein, (FIG. 7, center panel) spike protein, and (FIG. 7, right panel) membrane protein. As shown in FIG. 7, the Htet1/N, Htet1/S** and Htet1/M cell lines display doxycycline-inducible expression of the SARS-CoV-2 N, S**, and M proteins. Predicted molecular weight (MW) for primary translation product of N is 46 kDa, of S** is 141 kDa, and of M is 25 kDa. The high MW forms of M apparent in these experiments were evident whenever M was expressed on its own without the co-expression of other SARS-CoV-2 structural proteins.

To determine whether these cell lines could be used to interrogate patient plasmas for SARS-CoV-2 antibodies, each of the tester cell lines (Htet1/N, Htet1/S**, and Htet1/M) were mixed with a negative control cell line (Htet1, at ~10% of the cell population), seeded onto 96 well, glass-bottom plates, and incubated overnight in doxycycline-containing media.

The cells were fixed, permeabilized, and processed for immunofluorescence microscopy using human patient plasmas as source of primary antibody. Fluorescent anti-human Ig antibodies, and DAPI were used to capture plasma antibodies to the SARS-CoV-2 N, S, and M proteins by the Htet1/N, Htet1/S** and Htet1/M cell lines; and fluorescence micrographs of mixtures of Htet1 cells (~20% of cells), which did not express mCherry, and Htet1/N, Htet1/S**, and Htet1/M cells, each of which expressed mCherry were analyzed.

Of the 40 pre-COVID control plasmas that were tested, none showed any sign of specific reactivity with cells expressing the N, S**, or M proteins. This was not surprising, as these plasmas were collected prior to the COVID-19 pandemic, and also, because a positive signal in this assay must match the known subcellular distribution of the test proteins (N is nuclear excluded, S** is primarily at the plasma membrane, and M accumulates in intracellular compartments).

These tester cell lines were next used to interrogate 30 plasmas from hospitalized, PCR-confirmed, COVID-19 patients. These plasmas had been collected on the day of admission of the patient into the Johns Hopkins Hospital, all between April 7 and Apr. 22, 2020. As outlined above, Htet1/N, Htet1/S**, and Htet1/M cells were mixed with a small percentage of negative control cells (Htet1), followed by visual examination and digital image capture. Of the 30 COVID-19 patient plasmas that were tested, 23 scored positive for anti-N antibodies, 20 scored positive for anti-S antibodies, and 13 scored positive for anti-M antibodies. Moreover, anti-S antibodies were only detected in patients that had anti-N antibodies, and anti-M antibodies were only detected in patients that had both anti-N and anti-S antibodies (Table 1).

The anti-spike serology test described above employed a form of spike, S**, that was deliberately constrained to a single structural conformation by six amino acid changes. These mutations constrain spike to a trimeric, prefusion conformation, which is presumed to be form of spike of greatest immunological importance, as antibodies to this form of spike may have a higher likelihood of blocking infection in virus neutralizing assays. While logical, there is little support for the hypothesis that antibodies to this particular form of spike are the most likely to correlate with disease course or protection against future infection. Furthermore, antibody responses to other conformational forms of spike may also be protective from infection and/or disease. Therefore, whether a microscopy-based serology test might be able to detect patient immune responses to conformationally distinct forms of spike, especially if those forms are directed to different compartments of the cell was tested. The Htet1/SW1 cell line, which inducibly expressed the spike protein encoded by the original isolate of SARS-CoV-2 was generated, and processed for immunofluorescence microscopy using a small subset of COVID-19 patient plasmas and an antibody to the Golgi marker protein GM130. Fluorescence micrographs of cells expressing the Wuhan-1 isolate form of S (Htet1/SW1) or the D614G mutant form of spike (Htet1/SD614G) were analyzed. Cells were stained using human plasma Igs, antibodies specific for the Golgi marker GM130, and DAPI (human plasmas display variable reactivity towards different subpopulations of spike proteins located within the Golgi, the plasma membrane, and a large intracellular compartment). Human plasmas revealed antigenically distinct forms of spike in different compartments of the cell.

These experiments revealed that certain patient plasmas (i.e. E12 and E9) contained anti-spike antibodies that preferentially recognized forms of SW1 that are located at the plasma membrane and Golgi, similar to their reactivity towards S**. However, other plasmas (i.e. X5 and G4) preferentially recognized a distinct subpopulation of SW1 proteins located in large, non-Golgi, intracellular compartments. Taken together, these results indicated that COVID-19 patients generate distinct sets of antibody responses to multiple forms of SW1 that are expressed in human cells, and that some of these antigenically distinct forms of spike are located within different intracellular compartments.

Several studies have demonstrated that the D614G mutation is associated with increased transmission, elevated viral load, increased viral titers, and elevated levels of spike on nascent virions. However, it was unclear how this one amino acid substation in the S1 region of spike results in such dramatic changes in SARS-CoV-2 biology. To explore the possibility that this mutation generates these changes through an alteration in spike protein trafficking, parallel experiments with cells expressing SD614G were performed. These experiments revealed a subtle yet significant change in the subcellular distribution of SD614G, relative to SW1, reflected here in an apparent increase in spike protein accumulation within in large, non-Golgi, intracellular compartments and a reduction in Golgi-localized spike.

The sorting of spike to these large intracellular compartments, and the enhancement of this sorting by the D614G mutation, led to co-stain Htet1/SD614G cells for immunofluorescence microscopy with plasma G4 and a series of antibodies specific for marker proteins of the ER, ER-Golgi-intermediate compartment (ERGIC), Golgi, plasma membrane, and lysosome. Htet1/SD614G cells were processed for immunofluorescence microscopy using plasma G4, antibodies specific for (A) Lamp1, (B) Lamp2, (C) CD63/Lamp3, (D) mTOR, (E) calnexin, (F) Grp78, (G) ERGIC53, (H) ERGIC3, (I) GM130, and (J) CD81, and DAPI. The resulting images revealed that that large intracellular structures containing SD614G were lysosomes, based on the co-localization of spike with Lamp1, Lamp2, CD63/Lamp3, and mTOR. There was some amount of SD614G in other organelles of the secretory pathway, these large lysosome-related structures were not enriched for the ER proteins calnexin and BiP, the ERGIC proteins ERGIC53 and ERGIC3, or the plasma membrane/exosomal protein CD81. Together with prior observations, these results demonstrated that human cells traffic SARS-CoV-2 spike to lysosomes and provided strong evidence that the D614G mutation enhances the lysosomal sorting of spike.

The lysosomal form(s) of spike appear to be at least somewhat antigenically distinct, making it difficult to know the extent to which the preceding results reflect differences in spike protein sorting as opposed to differences in recognition of spike isoforms by the antibodies in different patient plasmas. To explore this issue it was first necessary to generate an anti-spike antibody that had the potential to detect different forms of spike, regardless of the extensive post-translational modifications and conformational variations that may have occurred in its large extracellular domain. An antibody to the peptide DSEPVLKGVKLHYTCOOH (SEQ ID NO: 1), which corresponds to the short, cytoplasmic, carboxy-terminal tail of spike, which is separated from its large extracellular domain by a lipid bilayer, was generated. This antibody was affinity purified, confirmed to be specific for spike (FIG. 7), and used to interrogate the intracellular distribution of both SD614G and SW1 by immunofluorescence microscopy.

Fluorescence micrographs of cells expressing the G614 form of spike (Htet1/SD614G) or the D614 form of spike (Htet1/SW1) were analyzed. Cells were stained using a rabbit antibody specific for the C-terminal 14 amino acids of spike, antibodies specific for GM130, Lamp1, or Lamp2, and DAPI. These experiments demonstrated that a significant proportion of spike proteins recognized by this anti-C-terminal antibody detected spike in lysosomes, regardless of whether the cells were expressing SD614G or SW1. Furthermore, they confirmed that the D614G mutation altered the subcellular trafficking of spike, a shift that was evident in reduced co-localization with the Golgi marker GM130, and an increase in its co-localization with the lysosomal markers Lamp1 and Lamp2. To quantify this effect, the percentage of Htet1/SW1 and Htet1/SD614G cells in which spike was found to co-localize with Lamp2 or GM130 was counted (Table 2). These data revealed that the D614G mutation causes an ~2-fold increase in this ratio (Table 2). It should be noted that this assay likely underestimates the magnitude of the D614G-mediated shift in spike protein distribution, as cells were scored as showing co-localization regardless of its extent.

In addition to demonstrating that spike was localized to lysosomes, the preceding experiments revealed that lysosomes appeared to be clustered in spike-expressing cells. To quantify this effect, Htet1, Htet1/SW1, and Htet1/ SD614G cells were stained with antibodies specific for Lamp2. These experiments revealed a low rate of lysosome clustering in Htet1 cells but much higher rates in cells induced to express SW1 or SD614G. Fluorescence micrographs of Htet1 cells, Htet1/SW1 cells, or Htet1/SD614G cells were analyzed. Cells were stained using plasma G4, antibodies specific for Lamp2, and DAPI. Counting cells in each population with Lamp2-positive clusters revealed that this occurred in <5% of Htet1 cells (9/355 cells) but increased to 8% after one day of SW1 expression (8/101 cells), and to 34% after three days of spike expression (21/62 cells). These experiments also revealed that the D614G mutation enhanced spike-induced lysosome clustering, as one day of SD614G expression led to lysosome clustering in 29% of cells (35/120), which increased to 51% of cells after three days of SD614G expression (61/119) (Table 3).

Given the correlation between the D614G mutation, enhanced SARS-CoV-2 transmission, and lysosomal trafficking & clustering, the reactivity of COVID-19 patient plasmas in an S1 ELISA test and a SD614G-based microscopy test were compared. It was found that COVID-19 patient plasmas displayed a wide array of reactivities in an anti-S1 ELISA test, spanning more than two orders of magnitude (see FIG. 8).

Interestingly, when these same samples were interrogated using the SD614G-based microscopy test, it was found that the strength of plasma reactivity in the anti-S 1 ELISA assay correlated relatively well with plasma membrane staining of SD614G-expressing cells but bore little or no relation to the strength of staining of lysosome-localized forms of SD614G.

Fluorescence micrographs of Htet1/SD614G cells stained with patient plasmas and an anti-Lamp2 antibody were analyzed. Anti-S1 antibody levels in 20 COVID-19 patient plasmas as evaluated by ELISA did not correlate antibodies to the lysosomal form of spike.

In conclusion, it appeared that the SD614G-based microscopy test generates an independent measure of anti-spike immune responses that is distinct from an anti-S1 ELISA assay, and therefore warrants further investigation for potential correlations with COVID-19 course of disease, response to treatment, and/or response to vaccination.

### Example 7

The data presented here demonstrated that microscopy-based SARS-CoV-2 serology assays can generate multidimensional outputs that incorporate signal strength, signal pattern specificity, and non-reactivity to internal negative controls. Moreover, it was establish that microscopy-based serology assays have the potential to report on conformational variations in the target protein, especially if those conformational variations impact the intracellular trafficking of the target protein. Such sensitivities are tied to the technological foundation of the microscopy-based serology assay, which interrogates immune responses to target proteins expressed in their native state, within their expressing cell, and without any extraction or modification other than chemically mild fixation. As such, the sensitivity of the microscopy-based serology platform cannot be matched by other technologies. In addition, microscopy-based assays such as those described in this report can be multiplexed for the simultaneous detection of immune responses to multiple target proteins in each sample. As for the scalability of microscopy-based serology tests, every step can be performed in a high-throughput, automated fashion, further reducing sources of error that may arise from sample handling. As for the clinical utility of these tests, they have the potential for lower false positive and negative results than unidimensional assays such as ELISA and have the potential for identifying clinically relevant information that is simply beyond the abilities of alternative testing technologies. In this context, it will be particularly interesting to determine whether immune responses to lysosome-localized and plasma membrane-localized forms of SD614G display important positive or negative correlations with course of COVID-19 disease, responses to treatments, and/or responses to different vaccines.

Data presented herein also revealed that SARS-CoV-2 spike is trafficked to lysosomes, that spike expression induces lysosome clustering, and that the D614G mutations enhanced the lysosomal localization of spike and spike-induced lysosome clustering. It was recently reported that lysosomes mediate the egress of another betacoronavirus, mouse hepatitis virus (MHV), raising the possibility that the lysosomal sorting of spike might promote its assembly into nascent SARS-CoV-2 virions. Such a model is attractive due to the consonant observations that the D614G mutation leads to increased trafficking of spike to lysosomes, and enhanced loading of spike into SARS-CoV-2 virions. However, these same observations can also be explained by a slightly different model in which SARS-CoV-2 virions assemble in other organelle (ERGIC, Golgi, etc.) but are diverted to lysosomes by spike-mediated and D614G-enhanced sorting of fully formed virus particles into lysosomes.

These two models are attractive due to their simplicity and the direct effect proposed for the D614G mutation on virus assembly and/or egress. However, the observations described herein were also consistent with a model in which the lysosomal sorting of spike regulates lysosome-related signaling pathways. More specifically, it was observed that spike expression and the D614G mutation both promote lysosome clustering, a phenomenon that is associated with alterations in lysosome function and lysosome-related signaling pathways such as AMPK and mTOR.

### Example 8

### Materials and Methods

### Cell Lines, Cell Culture and Transfections

HEK293 cells (ATCC) were cultured in complete medium (DMEM containing 10% fetal bovine serum and 1% penicillin/streptomycin solution). Transfections were carried out using lipofectamine 3000 according to the manufacturer’s instructions.

Htet1 cells were generated by transfecting HEK293 cells with the plasmid pS147, which encodes the tet-activated transcription factor rtTAv16, expressed from the CMV promoter as a bicistronic ORF encoding (a) rtTAv16, (b) a viral 2a peptide, and (c) a bleomycin-resistance protein, BleoR, followed by selection of zeocin-resistant transgenic cell clones (200 ug/ml zeocin), and pooling of these clones.

F/S121 cells expressing mCherry were generated by transfecting 293F cells with the Sleeping Beauty vector pS121, followed by selection and pooling of puromycin-resistant cell clones. SARS-CoV-2 protein-expressing cell lines were generated by transfection of Htet1 cells with Sleeping Beauty transposons that carry (a) a tet-regulated transgene designed to express one or another SARS-CoV-2 protein under control of the TRE3G promoter and (b) puromycin-resistance gene, followed by selection of puromycin-resistant (1 ug/ml puromycin) and zeocin-resistant (200 ug/ml zeocin) cell clones, followed by pooling of clones to generate individual cell lines. Tet-inducible gene expression was induced by addition of doxycycline to the culture medium at a concentration of 1 ug/ml for a period of 1-2 days.

### Plasmids

The plasmid used to create Htet1 cells was pS147, a CMV-based vector designed to express the rtTAv16 protein from a polycistronic ORF, upstream of a viral 2a peptide and the Bleomycin resistance coding region. Other vectors used in this study were based on a Sleeping Beauty transposon vector (pITRSB) in which genes of interest can be inserted between the left and right inverted tandem repeats (ITRs). These include three plasmids in which the region between the ITRs contains (a) one gene in which a crippled EF1alpha promoter drives expression of a polycistronic ORF encoding mCherry, the p2a peptide, and the puromycin-resistance protein, and (b) a second gene in which the TRE3G promoter drives expression of codon-optimized forms of the N, S**, or M proteins (pCG217, pCG218, and pCG221, respectively).

Two additional transposon-mobilizing plasmids were also used in this study, which contain (a) one gene in which a crippled EF1alpha promoter drives expression of the puromycin-resistance protein, and (b) a second gene in which the TRE3G promoter drives expression of codon-optimized forms of the SW1 and SD614G proteins (pCG145 and pCG200, respectively). In addition, we created pS121, a Sleeping Beauty transposon vector carrying a single gene between its ITRs, which consists of a CMV promoter upstream of a polycistronic ORF encoding mCherry, a viral 2a peptide, and a fusion protein between the destabilization domain of DHFR and the puromycin resistance protein.

### Immunoblot

HEK293 cell lines were grown in the presence or absence of doxycycline for a period of 2 days. Cells were then lysed by addition of sample buffer, separated by SDS-PAGE, transferred to PVDF membranes, and incubated with rabbit antibodies raised against the C-terminal peptides of the SARS-CoV-2 N protein, the SARS-CoV-2 S protein, and the SARS M protein. Following extensive washes, the membranes were probed using HRP-conjugated anti-rabbit antibodies, washed again, developed using chemiluminescence reagents, and visualized using a GE imaging system.

### Immunofluorescence Microscopy

Cells were cultured on either sterile, poly-L-lysine-coated cover glasses, or sterile, poly-L-lysine-coated, glass-bottom, black-walled 96 well plates. For serology testing, SARS-CoV-2 protein-expressing cells were mixed with the parental Htet1 cell line at a ratio of ~80%:20%. Cells were exposed to 1 ug/ml doxycycline for 1 day to induce SARS-CoV-2 protein expression. Cells were then fixed (4% formaldehyde in PBS), permeabilized (1% Triton X-100 in PBS), and processed for immunofluorescence microscopy. This involved incubating one side of a coverglass with primary antibodies or patient plasmas, followed by extensive washing, incubation with fluorescently labeled secondary antibodies, additional washes, and mounting on glass slide. Stained cells were visualized using an EVOS7000 fluorescence microscope (ThermoFisher) equipped with 20x, 40x and 60x objectives. Images were processed using Adobe Photoshop and assembled in Adobe Illustrator.

### Elisa

RayBio COVID-19 S1 RBD protein Human IgG ELISA kit (Catalog# IEQ-CoVS1RBD-IgG) was used for IgG antibody testing. PCR-confirmed Human COVID-19 plasma samples (diluted 1:1000) and negative and positive controls were added to the wells of S1RBD-coated plates (3 technical replicates/sample) in a total volume of 100 µls per well, and plates were incubated at 24° C. for 60 min on a shaker (200 rpm). After 4 wash steps with 1x washing buffer, 100 µls of diluted biotinylated anti-human IgG antibody was added to the wells (diluted 1:100), and samples were incubated at 24° C. for 30 min on a shaker (200 rpm). After 4 wash steps with 1x washing buffer, 100 µls of diluted HRP-Streptavidin solution was added to the wells (diluted 1:800), and samples were incubated at 24° C. for 30 min on a shaker (200 rpm). After 4 wash steps with 1x washing buffer, 100 µls of TMB substrate solution was were added, and samples were incubated at 24° C. for 15 min on a shaker (200 rpm). The reaction was terminated by adding 50 µls of Stop Solution (0.2 M sulfuric acid), and A450 was measured.

### Antibodies

Rabbit polyclonal antibodies to the SARS-CoV-2 proteins were a gift from C. Machamer, JHU. Rabbit polyclonal antibodies directed against ERGIC3, ERGIC53, Calnexin, and GRP78/BiP were obtained from ThermoFisher. Mouise monoclonal antibodies to Lamp1, Lamp2, Lamp3/CD63, CD9, CD81, calnexin, and GM130 were also obtained from ThermoFisher. Rabbit antibodies to mTOR were obtained from Cell Signaling. Fluorescently labeled (Alexa488, Alexa647, or Cy5) antibodies specific for human, rabbit, or mouse IgGs were obtained from Jackson Immunoresearch.

### Human Plasmas

Plasma simples were obtained from control subjects, and from subject with confirmed COVID-19 diagnosis.

Although the invention has been described with reference to the above examples, it will be understood that modifications and variations are encompassed within the spirit and scope of the invention. Accordingly, the invention is limited only by the following claims.

What is claimed is:

1. A method of detecting the presence of a target specific antibody in a sample comprising:
(i) contacting the sample with a test cell comprising one or more exogenous nucleic acid sequences encoding one or more target proteins; and
(ii) detecting the presence of the target specific antibody in the sample by contacting the immune complex of (i) with an anti-immunoglobulin (Ig) antibody, and detecting the anti-immunoglobulin (Ig) antibody, thereby detecting the presence of a target specific antibody.

2. The method of claim 1, further comprising contacting the sample with a control cell, wherein a target specific antibody present in the sample forms an immune complex with the one or more target proteins expressed by the test cell.

3. (canceled)

4. The method of claim 1, wherein the target protein is a pathogen protein.

5-6. (canceled)

7. The method of claim 4, wherein the pathogen protein is selected from SARS-CoV-2 spike (S), nucleocapsid (N), membrane (M), and envelope (E) proteins.

8. The method of claim 7, wherein the SARS-CoV-2 S protein is an SW1 protein, an SD614G protein or an S** protein.

9-10. (canceled)

11. The method of claim 1, wherein the anti-Ig antibody is a detectably labeled anti IgG, IgM, or IgA antibody, or a combination thereof.

12. (canceled)

13. The method of claim 11, wherein the anti-IgG, -IgM, or -IgA antibody detects an immune complex comprising a target protein expressed by the test cell and an anti-target specific antibody present in the sample.

14. (canceled)

15. The method of claim 13, wherein detecting an anti-target specific antibody in the sample comprises detecting the detectably labeled anti-Ig antibody and the detectable protein in the test cell but not in the control cell.

16. The method of claim 4, wherein detecting an anti-pathogen specific antibody in the sample indicates that the subject is infected by the pathogen, has developed an immunity against a pathogen related disease and/or infection, and/or is vaccinated against the pathogen related disease and/or infection.

17. The method of claim 13, wherein detecting the detectably labeled anti-Ig antibody alone in a test cell, detecting the detectable protein alone in the test cell, or detecting the detectably labeled anti-Ig antibody and/or the detectable protein in a control cell indicates an absence of anti-pathogen specific antibody in the sample.

18-21. (canceled)

22. The method of claim 2, wherein the test cell and control cell are present in a ratio of about 9:1 to 4:1.

23. (canceled)

24. The method of claim 1, further comprising detecting a sub-cellular localization of the target specific antibody, wherein detecting the subcellular localization of the target specific antibody comprises contacting the cells with one or more organelle-specific antibodies and determining co-localization of the target specific antibody and the organelle-specific antibodies.

25-31. (canceled)

32. The method of claim 24, further comprising detecting a subcellular localization of a target specific antibody in a second test cell, wherein the target protein in a first test cell and in the second test cell localize differently.

33. The method of claim 32, wherein the first and second target proteins are mutant variants of one another.

34. The method of claim 27, wherein the sample is collected from a subject, the target protein is SARS-CoV-2 Spike protein a SW1 protein, a SD614G protein or a S** protein, and detecting sub-cellular localization of the target specific antibody comprises the detection of localization to the Golgi, wherein localization to the Golgi indicates the presence of SARS-CoV-2 neutralizing antibodies in the sample.

35. The method of claim 1, wherein the target specific antibody is detectably labeled.

36. An isolated peptide of SEQ ID NO: 1 DSEPVLKGVKLHYT.

37. An antibody that specifically binds to the peptide of claim 36.

38. A kit comprising:
(i) a test cell comprising an exogenous nucleic acid sequence encoding a SARS-CoV-2 protein;
(ii) a control cell, and optionally
(iii) a detectably labeled anti-Ig antibody,
(iv) an anti-lysosome marker antibody, and/or
(v) an anti-Golgi marker antibody.

39. The kit of claim 38, wherein the test cell and the control cell are selected from the group consisting of an adherent fixed and permeabilized cell, a suspension of fixed and permeabilized cell and a cell lysate coated on a surface.

40-46. (canceled)


SEQUENCE LISTING (1 sequence)

SEQ ID NO: 1 - protein, 14 aa, Artificial Sequence
  Description of Artificial Sequence: Synthetic peptide
DSEPVLKGVKLHYT